(19) United States
(12) Patent Application Publication
(10) Pub. No.: US 2026/0072012 A1
(43) Pub. Date: Mar. 12, 2026

(54) CELL BASED PROTEIN DEGRADATION SCREENING ASSAY AND KITS THEREOF

(71) Applicant: Eurofins DiscoverX Corporation, San Diego, CA (US)

(72) Inventors: Alexander Michael Clifford, San Diego, CA (US); Jean Alexandre Bernatchez, San Diego, CA (US)

(21) Appl. No.: 19/318,331

(22) Filed: Sep. 3, 2025

Related U.S. Application Data
(60) Provisional application No. 63/692,679, filed on Sep. 9, 2024.

(51) Int. Cl. G01N 33/50; G01N 33/58

(52) U.S. Cl. CPC G01N 33/5023; G01N 33/58

(57) ABSTRACT

A cell based compound screening method to determine degradation properties and kinetics of a test compound comprising screening the test compound that binds both a target protein of interest and a ligase to effect degradation, wherein the test compound is not previously known to degrade the target protein of interest.

### PRIORITY CLAIM AND CROSS-REFERENCE TO RELATED APPLICATIONS

This application claims the benefit and priority of the U.S. Provisional Application No. 63/692,679, filed on Sep. 9, 2024, the disclosure of which is incorporated by reference herein in its entirety, including all references and appendices cited therein, for all purposes.

### FIELD OF INVENTION

The subject matter provided herein relates to a protein degradation assay using affinity capture technology and methods of practicing the same. Also provided herein are kits and methods of use thereof.

### BACKGROUND

Drug discovery techniques have primarily focused on inhibiting protein function through small molecule binding either to an active site or an allosteric site. However, such methods face difficulties as many disease-relevant proteins lack druggable binding pockets or are considered “undruggable”. Additionally, protein inhibition may not always provide the desired therapeutic outcome, as inhibited proteins can still retain some biological functions.

Targeted protein degradation has emerged as a promising therapeutic modality that can address limitations of traditional inhibition-based approaches. This strategy involves recruiting cellular protein degradation machinery to selectively degrade disease-causing proteins.

Current methods for identifying compounds capable of inducing targeted protein degradation often rely on prior knowledge of degradation activity or require extensive optimization of known degrader scaffolds. However, there remains a need for systematic screening approaches that can identify novel compounds with previously unknown protein degradation capabilities.

Existing screening methods may not provide comprehensive kinetic information about degradation processes, limiting the ability to optimize compounds for desired degradation profiles. There is a need for improved screening methodologies that can efficiently identify and characterize novel protein degraders while providing detailed kinetic information about their degradation properties.

### SUMMARY

The following is a summary of the invention to provide a basic understanding of some aspects of the invention. This summary is not intended to identify key/critical elements of the invention or delineate the invention's scope. Its sole purpose is to present some concepts of the invention in a simplified form as a prelude to the more detailed description that is presented later.

The present disclosure provides methods and kits for screening a test compound as a protein degrader and assessing the degradation kinetics of such a test compound using affinity capture technology.

In various embodiments, the present disclosure provides a method of assessing the degradation activity of a test compound, the method comprising contacting a cell with the test compound, the cell comprising a fusion protein comprising a target protein of interest and a DNA-binding protein domain, incubating the cell under conditions promoting degradation of the target protein of interest, lysing the cell to form cell lysate, linking the fusion protein in the lysate to a nucleic acid oligomer tag, incubating the cell lysate comprising the fusion protein linked to the nucleic acid oligomer tag in the presence of a ligand immobilized on a solid support, wherein the ligand is known to bind to the target protein of interest. The method further comprises removing cellular proteins that do not exhibit binding to the ligand, wherein said cellular proteins lack any known binding interaction with the ligand, and detecting an expression level of the target protein of interest bound to the ligand by detecting the nucleic acid oligomer tag, wherein the expression level of the target protein of interest from lysates harvested from cells incubated in the presence of the test compound is compared to the expression level of the target protein of interest from lysates harvested from cells incubated in the absence of the test compound, wherein a decrease in the expression level of the target protein of interest in the presence of the test compound as compared to the absence of the test compound indicates the test compound promotes degradation of the target protein of interest. In many embodiments, the test compound is not previously known to degrade the target protein of interest. In many other embodiments, the target protein of interest and the nucleic acid oligomer tag are not the same.

In various embodiments, the disclosure provides a method of assessing a degradation activity of a test compound, comprising contacting a cell comprising the fusion protein with a control compound, wherein the control compound is previously known to degrade the target protein of interest, lysing the cell to form cell lysate, linking the fusion protein in the lysate to a nucleic acid oligomer tag, incubating the cell lysate comprising the fusion protein linked to the nucleic acid oligomer tag in the presence of a ligand immobilized on a solid support, detecting the expression level of the target protein of interest by detecting the nucleic acid oligomer tag; and then comparing the expression level of the target protein of interest bound to the ligand in the absence of the test compound to the expression level of the target protein of interest bound to the ligand in the presence of the test compound, wherein a decrease in the expression level of the target protein of interest in the presence of the test compound as compared to the expression level of the target protein of interest in the absence of the test compound indicates the test compound promotes degradation of the target protein of interest. In various embodiments, the control compound is a control degrader, wherein the degrader is a known degrader for the target protein of interest and therefore, the presence of the control degrader in the assay would decrease the expression level of the target protein of interest as compared to the assay where the cells containing the target protein of interest are incubated in the absence of test compound.

In various embodiments, the test compound is not previously known to degrade the target protein of interest, and therefore, the assay assesses the test compound for its degradation properties as to whether the test compound can bind the target protein of interest, tag the target protein of interest for degradation wherein a ligase, such as an E3 ligase, binds to the target protein of interest and tags the target protein of interest through ubiquitination and subsequent degradation within a proteosome. The method further helps assess the degradation kinetics of the test compound by measuring the amount of degradation in a given period of time.

In many embodiments, the method further comprises contacting an expression vector comprising the fusion protein with the test compound. In many other embodiments, the target protein of interest bound to the ligand is an intact target protein of interest or a target protein of interest.

In various embodiments, the method further comprises contacting the cell with an inhibitor previously known to inhibit protein degradation.

In many embodiments, the present disclosure provides a method of assessing a degradation activity of a test compound, comprising contacting a cell simultaneously with the test compound and an inhibitor previously known to inhibit protein degradation, the cell comprising a fusion protein, wherein the fusion protein comprises a target protein of interest and a DNA-binding protein domain, incubating the cell under conditions promoting the degradation of the target protein of interest, lysing the cell to form cell lysate, linking the fusion protein in the lysate to a nucleic acid oligomer tag, incubating the tagged cell lysate comprising the fusion protein linked to the nucleic acid oligomer tag in the presence of a ligand immobilized on a solid support, wherein the ligand is known to bind to the target protein of interest, removing cellular proteins that do not exhibit binding to the ligand, wherein said cellular proteins lack any known binding interaction with the ligand, detecting an expression level of the target protein of interest bound to the ligand by detecting the nucleic acid oligomer tag in lysates from cells incubated in the presence of the test compound and the inhibitor, and comparing with an expression level of the target protein of interest in lysates from cells incubated in the presence of test compound and in the absence of the inhibitor, wherein an increase in the expression level of the target protein of interest in the presence of the inhibitor and the test compound as compared to in the absence of the inhibitor, indicates that the test compound reduces the expression level of the target protein of interest by promoting protein degradation.

In many embodiments, the inhibitor is previously known to inhibit the degradation of the target protein of interest. In many embodiments, the inhibitor may be a ubiquitination inhibitor, a protein inhibitor, or a proteosome inhibitor.

In various embodiments, a reference ligand may be immobilized on a solid support such as a bead. The bead may be a magnetic bead or a streptavidin bead. In various other embodiments, the ligand is immobilized on streptavidin-coated magnetic beads, which may be further treated with biotinylated small molecule ligand to generate affinity resins for the disclosed assays. The ligand may be a biotinylated ligand. In various other embodiments, a ligand, such as ligand immobilized on a solid support may be a protein, a small molecule, or a peptide, wherein the protein is immobilized on a solid phase support bead wherein the protein will be cloned with an Avi tag at its N-terminus followed by purification and labelling with Desthio-Biotin, and then the labelled protein is immobilized to a magnetic streptavidin bead.

In various embodiments, the ligand may be labelled with a tag for detection. The tag may be an enzymatic tag, a fluorescent tag, a spectroscopic tag, a reporter group, a fluorescent probe, or other detection probes.

In various embodiments, the test compound may be a test molecule, a heterobifunctional degrader, a molecular glue, a small molecule, a drug, a biologic, a protein, a pharmaceutical drug, a candidate pharmaceutical, a carbohydrate, or other compounds.

In many embodiments, the detection comprises quantifying the phage by quantitative PCR of an amplicon located within the phage genome or via other detection techniques known for detecting fluorescent tag, spectroscopic tag, enzymatic probe and other detection methods.

The disclosed assay may be conducted within a column, a single-well plate, or a multi-well plate.

The present disclosure also provides screening libraries of test compounds against the protein or protein domain of interest, and once the test compound is identified as a protein degrader, the test compound can be further evaluated individually.

The disclosure further provides methods of quantifying the interaction between phage-displayed protein or protein domain and test compound. Also included are business methods for the pharmaceutical development of test compounds screened using the disclosed assay.

These and other features, aspects, and advantages of the present systems and methods will become better understood with references to the following figures and descriptions. This summary is an introduction to the concepts. Additional aspects and advantages of this invention will be apparent from the following detailed description of preferred embodiments, which proceeds with reference to the accompanying drawings.

### BRIEF DESCRIPTION OF THE FIGURES

The accompanying drawings, where like reference numerals refer to identical or functionally similar elements throughout the separate views, together with the detailed description below, are incorporated in and form part of the specification and serve to illustrate further embodiments of concepts that include the claimed disclosure, and explain various advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 shows the disclosed screening assay principle.

FIGS. 2A-2B depict Assayed profiles of CRBN-mediated degradation (STAT3xSD36, IRAK4xKTX-951) in the presence of vehicle control (in dashed line) or in the presence of known proteasomal (in solid line) or neddylation (in vertical drop lines with solid triangle) inhibitors. Profiles of negative control targets (STAT1xSD36 and IRAK1xKTX-951) are displayed in vertical drop lines with hollow triangles.

FIG. 3 depicts assayed profiles of VHL-mediated degradation (BCLx1×PZ703b) in the presence of vehicle control (in dashed line) or in the presence of known proteasomal (in solid line) or neddylation (in dash-dotted line) inhibitors. Profiles of the negative control target (BCLA1xPZ703b) are displayed in vertical drop lines with hollow triangles.

### DETAILED DESCRIPTION

The following embodiments provided herein are exemplary and are not limitations. The methods disclosed herein have a range of applications, all of which are based on the ability to detect and quantify a protein of interest for its degradation properties. The compositions and methods provided herein may be used in vitro and/or in vivo.

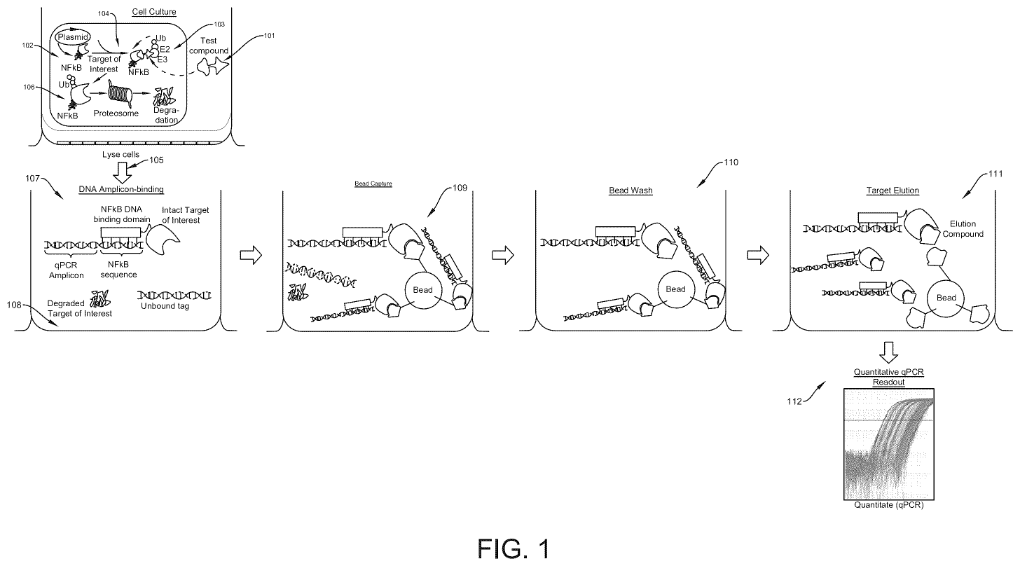

In many embodiments, the present disclosure provides a cell-based compound screening method allowing for a determination of degradation properties and kinetics of a test compound, wherein the test compound may be a heterobifunctional degrader or a molecular glue such that the test compound binding with a target protein of interest provides a binding site for a ligase such that a ligase binds to the target protein of interest and tags the target protein of interest for degradation (101). In various embodiments, the disclosed screening method comprises contacting a cell, the cell comprising a fusion protein and a test compound (102). The test compound has more than one binding site, such that the test compound binds to the target protein of interest and provides a binding site for a ligase to tag the target protein of interest for degradation (103). The method further comprises incubating a cell comprising the target protein of interest with the test compound (104) such that if the test compound is a protein degrader, the test compound will bind to the target protein of interest and provide a binding site for a ligase wherein the binding of the ligase will tag the protein for degradation. The cell is lysed (105) for measuring the target protein of interest expression in a cell lysate wherein if the test compound has degradation properties or affinity for the target protein of interest, the test compound will bind the target protein of interest and provide a binding site for a ligase, wherein binding of the ligase will tag the target protein of interest for degradation. Once the target protein of interest is tagged for degradation (106), the expression level of the target protein of interest will decrease in the cell extract as the protein of interest will degrade via proteosomal degradation. The fusion protein in the cell lysate is linked to a nucleic acid oligomer tag (107), and the tagged cell lysate is incubated in the presence of a ligand immobilized on a solid support, wherein the ligand is known to bind the target protein of interest (FIG. 1).

As further shown in FIG. 1, during incubation, the ligand will bind the target protein of interest present as an intact protein (109). Degraded (108) and non-specific proteins in the cell lysate are washed away from the solid support, and an elution compound is added to unbind the target protein of interest from the solid-support ligand (110). The expression level of the target protein of interest in the eluate or lysate is measured (111) to quantify the amount of target protein of interest bound to the ligand. If the test compound has an affinity for the target protein of interest and tags it for degradation, the measured expression level in the lysate of the target protein of interest will be lower compared to the level of the target protein of interest in the absence of the test compound. Conversely, if the test compound does not have an affinity for the target protein of interest and does not tag it for degradation, the expression level of the target protein of interest in the lysate will be higher compared to the level in the presence of the control compound. The expression level is a quantitative PCR readout (112).

In some embodiments, provided herein is a nucleic acid oligomer (tag) that binds a nucleic acid-interacting motif, wherein the nucleic acid oligomer comprises (a) a first nucleic acid sequence that is a PCR amplification sequence and (b) a second nucleic acid sequence that binds the nucleic acid-interacting motif, wherein the first nucleic acid sequence is heterologous to the second nucleic acid sequence.

In many embodiments, the control compound is tested simultaneously with the test compound and ensures that the assay is properly functioning. If the control compound displays a lower level of protein expression when added to the cells when compared to the absence of compound treatment, then a test compound that does not decrease the level of protein expression in the same experiment can be judged as having no effect on the target protein's expression level. Conversely, if a test compound decreases the expression level of the target protein of interest, it is a putative protein degrader of the target protein of interest since it performs like the control compound in the assay.

In one embodiment, the length of the nucleic acid oligomer is between about 50 and about 100, about 50 and about 200, about 50 and about 300, about 50 and about 400, about 50 and about 500, about 100 and about 200, about 100 and about 300, about 100 and about 400, about 100 and about 500, about 200 and about 300, about 200 and about 400, about 200 and about 500, about 300 and about 400, about 300 and about 500, or about 400 and about 500 nucleotides in length.

As used herein, the term “about” or “approximately” means within 20%, preferably within 10%, and more preferably within 5% (or 1% or less) of a given value or range.

In some embodiments, the nucleic acid tag has a reporter function and a protein tagging function. As used herein, a “reporter” function with reference to a nucleic acid tag is the ability to be visualized or otherwise detected or quantitated. In certain embodiments, the reporter function of a nucleic acid tag comes from the radiolabeling, fluorescent labeling or biotinylation of the nucleic acid tag. As used herein, a “nucleic acid tag” is a polynucleotide, e.g., an oligomer, that binds or is capable of binding to a target protein of interest, such as a protein fusion comprising a heterologous polynucleotide-binding domain (also called a polynucleotide-interacting motif herein), such as a DNA-binding domain. The nucleic acid tag may be single- or double-stranded DNA, single- or double-stranded RNA, DNA-RNA hybrid, RNA-RNA hybrid, or their native or synthetic derivatives, analogs and fragments thereof. In some embodiments, the nucleic acid tag is DNA, and the reporter function label can be introduced to the DNA, for example, by any standard enzymatic reaction, such as nick translation, or by terminal labeling, with 32P, 1251 or biotin-labeled deoxynucleotide triphosphates (dNTPs), or the label can be introduced as an intercalating agent. There are many fluorescent groups that are commercially available and can be used to label the nucleic acid tag. Some examples of fluorescent labels that can be used to label the nucleic acid tag are fluorescein isothiocyante, rhodamine and coumarin and their commercial derivatives, such as Texas Red® and Alexa Fluor®.

In certain embodiments, the nucleic acid tag is complexed, covalently linked or non-covalently linked to a detectable protein or polypeptide, for example, by a covalent linkage. Nucleic acid-protein fusions can be produced by any method, for example, by the method of Roberts and Szostak (U.S. Pat. Nos. 6,258,558 and 6,261,804; WO 98/31700; Roberts & Szostak (1997) Proc. Natl. Acad. Sci. USA (1997) 94:12297-12302) using a peptide acceptor, such as puromycin, as a covalent linking agent. Briefly, such an exemplary method comprises an in vitro or in situ transcription/translation protocol that generates a protein covalently linked to the 3′ end of its own mRNA, i.e., an RNA-protein fusion. This is accomplished by synthesis and in vitro or in situ translation of an mRNA molecule with a peptide acceptor attached to its 3′ end. In specific embodiments, the peptide acceptor is puromycin, a nucleoside analog that adds to the C-terminus of a growing peptide chain and terminates translation. In one embodiment, a DNA sequence is included between the end of the message and the peptide acceptor, which is designed to cause the ribosome to pause at the end of the open reading frame, providing additional time for the peptide acceptor (for example, puromycin) to accept the nascent peptide chain before hydrolysis of the peptidyl-tRNA linkage.

In other embodiments, the reporter function of a nucleic acid tag is a nucleic acid sequence that is amplifiable by PCR (also referred to herein as an “amplicon”). The amplifiable sequence hybridizes or is capable of hybridizing to a PCR primer in a sequence-specific manner. In certain embodiments, the nucleic acid tag comprises a plurality of amplicons, for example, two, three, four, five, six, seven, eight, nine, ten or more amplicons. In some embodiments, the plurality of amplicons are tandem repeats of a single amplicon. In certain embodiments, the amplicon is amplifiable by quantitative PCR, which permits the quantification of the protein tagged with such a nucleic acid tag. In a specific amplification method, amplification of a PCR sequence includes combining the nucleic acid containing the PCR amplification template, PCR primer and qPCR probe in a standard PCR reaction mixture (generally, a mixture having a final concentration of 10 mM Tris-HCl (pH 8.3 at 25° C.), 1-4 mM MgCl₂, 0.1-1 mM dNTP), and treating the sample first under Hot Start conditions (for example, heating to 95° C. for 5 minutes) to minimize nonspecific annealing or mispriming, followed by a denaturation step (for example, 95° C. for 45 seconds), followed by an annealing step (55° C. for 1 minute), and followed by an extension step (72° C. for 1 minute), with up to forty rounds of the consecutive steps of denaturation, annealing and extension, to complete the amplification of the qPCR signal.

As used herein, a “target protein of interest” can be any conceivable polypeptide or protein that may be of interest, such as to study or otherwise characterize. In some embodiments, the target protein of interest is a kinase, transcription factor, phosphatases, oxireductases, transferase, oxidoreductase, hydrolase, ligase, isomerase or lyase. In one embodiment, the target protein of interest is a human polypeptide or protein. In certain embodiments, the target protein of interest is a transferase having transferase activities, such as an acyltransferase, glycosyltransferase, amidotransferase or sulfurtransferase. In another embodiment, the target protein of interest is a hydrolase, peptidase, protease phosphatase, transport protein, storage protein, or regulatory protein.

In some embodiments, a target protein of interest is a transmembrane protein, such as a 7-transmembrane helix protein, such as a G-protein coupled receptor (GPCR). A target protein of interest may also be a transmembrane ion channel protein, and in certain embodiments, a ligand-gated ion channel protein. In other embodiments, a target protein of interest is a nuclear hormone receptor protein, such as a classic steroid hormone receptor and/or a receptor in the orphan class of nuclear hormone receptors.

In yet other embodiments, a target protein of interest is an extracellular signaling molecule or factor, such as a cytokine (e.g., an interferon and/or an interleukin), growth factor, and/or hormone (e.g., insulin, glucagon or prostaglandins). In certain embodiments, a target protein of interest is a protein involved in intracellular signal cascades, such as an enzyme or cofactor involved in phosphatidinyl-inositol signaling, cAMP, or cGMP generation.

In some embodiments, a target protein of interest is an antibody, small chain variable fragment (scFv), antigen or epitope.

The target protein of interest can, in some embodiments, be the expression of a nucleotide sequence generated by random mutation, the expression of a nucleotide sequence containing systematically synthesized sequences, or it may be an expressed cDNA. In one example, the target protein of interest being studied or characterized is derived from a human cDNA library (i.e., a human protein).

In certain embodiments, the target protein of interest is a chimeric fusion between a protein of interest and a heterologous DNA-binding protein. In such chimeric fusions, at least two gene sequences representing each half of the chimera can be fused in-frame, cloned into the appropriate vector and expressed in a host cell of choice. In certain embodiments, the target protein of interest is 5′ of the nucleotide-binding domain (e.g., DNA-binding protein). In other embodiments, the target protein of interest is 3′ of the nucleotide-binding domain (e.g., DNA-binding protein). In specific embodiments, the target protein of interest and/or the nucleotide-binding domain (e.g., DNA-binding protein) retain the respective activity of the wild-type protein. The target protein of interest, including chimeric fusions, may be expressed in any of a variety of host cells, including bacterial, insect, mammalian or plant host cells. When the target protein of interest is expressed in the appropriate eukaryotic host cell, it can exhibit post-translational eukaryotic modification that is present in the native protein and is, therefore, expected to have the structure and function of a native protein.

Also provided herein is a library of fusion proteins, comprising a plurality of fusion proteins provided herein, wherein at least two or more of the fusion proteins differ from each other. In certain embodiments, provided herein is a library of oligomers comprising a plurality of oligomers provided herein, wherein at least two or more of the oligomers differ from each other. Also provided herein is a nucleic acid encoding a fusion protein provided herein, as well as a vector comprising a nucleic acid encoding a fusion protein provided herein. Additionally, provided herein is a host cell comprising a vector comprising a nucleic acid encoding a fusion protein provided herein. In certain embodiments, the host cell is a bacterial, insect, mammalian or plant host cell.

In certain embodiments, also provided herein is a functional screening assay, which studies the degradation activity of the target protein of interest. In some embodiments, the activity of a target protein of interest is assessed using a nucleic acid tag, such as by detecting the presence of the nucleic acid tag in a given period of time at a defined concentration of the test compound.

The ligand may optionally be immobilized, such as by attachment to a solid support, e.g., a bead surface. The ligand may also be optionally labeled with, for example, fluorescence and/or spectroscopic tags. Alternatively, the reference moiety may be labeled with a reporter group, such as a fluorescent probe, that permits alternative readouts of the interaction between the protein domain and the ligand. Fluorescence polarization is a non-limiting example of a method that could be used to detect interactions between the labeled ligand and the protein domain at various concentrations of the test compound. The ligand may be a small molecule, a drug, a biologic, a protein, a carbohydrate, a pharmaceutical drug, a candidate pharmaceutical or other compounds.

The reference ligand (bait) can be captured using any standard procedure, for example, by biotinylation of the reference ligand, followed by capture of biotinylated reference ligand using immobilized streptavidin (for example, streptavidin immobilized on magnetic beads or a column). Proteins of interest that bind to the reference ligand (and nucleic acid tags, which bind to the proteins of interest) will remain bound to the solid support, while unbound binding reagents (proteins of interest and/or nucleic acid tags/cellular proteins/background proteins) are washed away. Following the capture of a bound protein of interest, a nucleic acid tag that has bound a target in the sample (e.g., or a protein of interest of a panel of proteins of interest) is detected simply by performing a PCR reaction using primers which hybridize to the amplicon portion of the nucleic acid tag. In certain embodiments, the PCR reaction is carried out using standard quantitative methods (for example, using Taq Man by Perkin-Elmer). In some embodiments, multiple protein of interest-nucleic acid tag complexes are retained by the solid support, in which case the individual members of the isolated pool can be identified, such as through the amplification of each unique nucleic acid tag, which is specific for a particular target protein of interest, e.g., in a panel.

Any of the screening assays described herein can be run in either singleplex or multiplex format. In one exemplary multiplex format, a test compound is simultaneously screened and tested for its degradation properties against multiple proteins from a panel of proteins of interest. Where multiple proteins of interest are being assayed simultaneously or sequentially, nucleic acid tags unique to each target protein of interest (e.g., different amplicons) can be used to distinguish the different proteins. For example, where the nucleic acid tag contains a PCR amplification marker, the PCR amplification marker would be unique to the particular target protein of interest to be detected. Each protein can, therefore, be tagged by a nucleic acid tag comprising a DNA target sequence and a PCR amplification marker that are each unique to the protein of interest. In this particular format, because each nucleic acid tag binds uniquely to a specific protein, the proteins of interest may be pooled at the elution step.

Also provided herein is a kit for screening test compounds as target protein of interest degraders. Such a kit may be comprised of a reference ligand (or “bait”), which is optionally immobilized onto a solid support or a container, such as a well in a multi-well plate; a detectable nucleic acid tag; and a protein of interest capable of being “tagged” by the nucleic acid tag. Where the nucleic acid tag is detectable by qPCR, the kit may additionally include a PCR primer capable of recognizing a PCR initiation sequence in the nucleic acid tag. Such a kit may be used to carry out the screening assay as described above.

### Experimentation

### Small Molecules and Nucleic Acids

PZ703b, SD-36, KTX-951, Bortezomib, and Pevonodistat were purchased from MedChemExpress (Monmouth Junction, NJ). The biotinylated affinity probes used for the affinity capture were custom-synthesized by SAI Life Sciences (Watertown, MA). DMSO was purchased from Sigma-Aldrich (St. Louis, MO). The DNA probe containing the PCR amplicon used to tag the NFκB fusion domain was custom-synthesized by Thermo Fisher Scientific (Waltham, MA). Test compounds were prepared as 1000× stocks in DMSO and rapidly diluted into the aqueous environment. DMSO was added to control assays lacking a test compound (0.2%).

### Protein Constructs

IRAK4, BCLxL, STAT3, IRAK1, STAT1 and BCLA1 constructs were designed as N-terminal fusions with the DNA-binding domain of NFkB (consisting of residues 35-36 fused to residues 41-359, using UniProt entry P19838 as a reference).

### Cell Culture and Transfection

HEK293T cells were maintained in T255 flasks in M3+PS (DMEM (Dulbecco's Modified Eagle Medium)+GlutaMAX-1, supplemented with 10% FBS (Fetal Bovine Serum)), 1% penicillin-streptomycin, and incubated at 37° C. with 5% CO₂. When the cells reached approximately 80% confluence, they were trypsinized using 0.25% Trypsin-EDTA and resuspended in a fresh culture medium.

Cells were seeded onto 12-well culture dishes, pretreated with Cultrex BME (R&D Biosystems; 1:100) at a density of 1.95× 10⁵ cells/well and allowed to adhere overnight under standard culture conditions in M3-PS (DMEM+GlutMAX+10% FBS).

The following day, the cells were transfected with a fusion construct using Lipofectamine 2000 according to the manufacturer's instructions. Briefly, the fusion construct DNA (100 ng per well) was diluted in Opti-MEM, and the transfection reagent (2.6 μL per well) was added to the DNA solution. The DNA-transfection reagent mixture was incubated at room temperature for 20 minutes to form DNA-lipid complexes, which were then added dropwise to the cells.

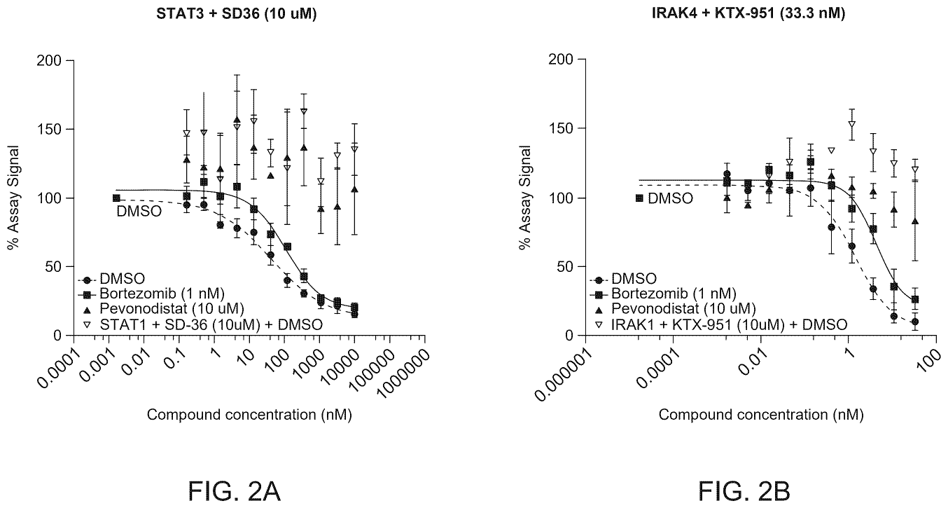

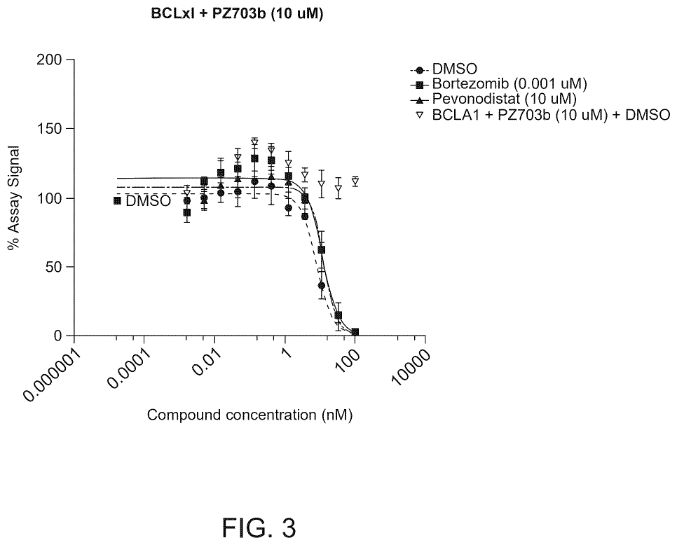

After 24 hours, the transfection medium was replaced with fresh M3-PS, and cells were then treated with an 11-point gradient of test compound (final top concentration: SD-36, 10 μM; KTX-951, 33.3 nM; PZ703b, 10 μM) or a combination of test compound and inhibitor of protein degradation compound (Bortezomib, 1 nM; Pevonodistat, 10 μM) for 18-24 hours. A subset of cells was treated with vehicle control alone (0.2% DMSO), see FIGS. 2A-2B and FIG. 3

### Cell Harvesting

Following compound treatment, the cells were washed once with room temperature cold PBS and then harvested via incubation in M-PER extraction buffer (Pierce Biotechnology, Rockford, IL) supplemented with 150 mM NaCl, 10 mM DTT, Protease Inhibitor Cocktail Complete (Roche Diagnostics GmbH, Mannheim, Germany) and Phosphatase Inhibitor Cocktail Set II (Merck KGaA, Darmstadt, Germany) following the manufacturer's guidelines. Lysates were collected, and cell debris was removed by passing extracts through filters (Pall AcroPrep 96 Filter Plate 3.0 μM Glass Filter/0.2 μM Bio-inert 1 mL well) under centrifugal force (3000×g), with the filtrate stored at −80° C. until further use.

### Bead Preparation

Preparation of liganded beads was performed as follows: the biotinylated affinity ligand (Bait peptide #1, Bait compound #1 and Bait compound #2) was incubated with streptavidin-coated magnetic beads (Thermo Fisher Scientific, Waltham, MA) for 30 minutes at 25° C. In order to remove the unbound affinity ligand and to reduce nonspecific binding of proteins in the cell lysate, the liganded beads were then blocked with excess biotin (125 nM) and washed with a blocking buffer containing SeaBlock (Pierce Biotechnology), 1% BSA and 0.05% Tween 20 and then stored in either Storage buffer 1 (Bait peptide #1) or Storage buffer 2 (Bait compound #1 and Bait compound #2).

### Affinity-Capture Quantitative Protein Expression Assay

Cell extracts were incubated in 96-well square-well microplates in the presence of a DNA-probe specific for the NFkB Binding domain expressed on the fusion constructs (1:80 dilution) in either tagging buffer 1 (STAT3/STAT1) or tagging buffer 2 (IRAK4/IRAK1 and BCLxL/BCLA1). DNA-tagged extracts were further diluted (1:100) in affinity capture buffer (1×PBS, 0.05% Tween 20, 1 mM DTT, 0.1% BSA, 2 mg/mL sheared salmon sperm DNA) in 96-well 2-mL natural polypropylene plates.

The affinity capture reaction was prepared with the tagged and blocked extract preps and the beads in deep well, natural polypropylene 384-well plates, catalog number 784201 (Greiner Bio-One, Kremsmünster, Austria) in a final volume of 19.5 μL.

No enzyme purification steps were performed on the protein extracts before adding them to the reaction mixture, and the protein extracts were diluted 8,000-fold in the final reaction mixture (the final DNA-tagged enzyme concentration was less than 0.1 nM). Capture assay mixtures were incubated at 25° C. with shaking for 1 hour.

After the incubation period, the affinity beads were extensively treated with wash buffer (1× PBS, 0.05% Tween 20) to remove unbound protein from the protein lysate. Using an elution buffer (1×PBS, 0.05% Tween 20 and either Elution compound 1, Elution cocktail 1, or Elution compound 2, the beads were resuspended and incubated at 25° C. while shaking for a 30-minutes period. The concentration of fusion protein in the eluates was then determined by quantitative PCR. DCso values for each degrader compound were determined using eleven compound doses in a serial threefold dilution.

### Analysis and Statistics

Data were normalized against DMSO-control values. Normalized data were used to calculate half-maximal degradation values (DC₅₀s) for each experiment using a standard dose-response curve fitting using the Hill Equation:

$$Response = Background + \frac{( Signal - Background )}{1 + {10}^{( log {DC}_{50} - [ degrader ] ) \times Hill Slope}}$$

The Hill Slope was set to 1. A non-linear least square fit using the Levenberg-Marquardt algorithm was employed for curve fitting. Data was plotted in GraphPad Prism Version 10.2.3 (San Diego, CA).

The ligand may optionally be immobilized, such as by attachment to a solid support, e.g., a bead surface. The ligand may also be optionally labeled with, for example, fluorescence and/or spectroscopic tags. Alternatively, the reference moiety may be labeled with a reporter group, such as a fluorescent probe, that permits alternative readouts of the interaction between the protein or protein domain and the ligand. Fluorescence polarization is a non-limiting example of a method that could be used to detect interactions between the labeled ligand and the protein domain at various concentrations of the test compound. The ligand may be a small molecule, a drug, a biologic, a protein, a carbohydrate, a pharmaceutical drug, a candidate pharmaceutical or other compounds.

The disclosure also provides kits for assessing degradation activity of a test compound, comprising a fusion protein comprising a target protein of interest and a DNA-binding protein domain, and instructions for the same.

The disclosure further provides a kit for assessing a degradation activity of a test compound, comprising (a) cells comprising a fusion protein, wherein the fusion protein comprises a target protein of interest and a DNA-binding protein domain; (b) nucleic acid oligomer tags configured to link to the fusion protein; (c) a solid support having immobilized thereon a ligand known to bind to the target protein of interest; (d) reagents for lysing the cells; (e) reagents for detecting the nucleic acid oligomer tags; and (f) instructions for: (i) contacting the cells with a test compound; (ii) incubating the cells under conditions promoting degradation of the target protein of interest; (iii) lysing the cells to form cell lysate; (iv) linking the fusion protein present in the lysate to the nucleic acid oligomer tags; (v) incubating the cell lysate in the presence of the immobilized ligand; (vi) detecting an expression level of the target protein of interest bound to the ligand by detecting the nucleic acid oligomer tags in the presence of the test compound; and (vii) comparing the expression level to that obtained in the absence of the test compound to determine if the test compound promotes degradation of the target protein of interest.

In the description, for purposes of explanation and not limitation, specific details are set forth, such as particular embodiments, systems, methods, etc. in order to provide a thorough understanding of the present technology. However, it will be apparent to one skilled in the art that the present technology may be practiced in other embodiments that depart from these specific details.

While the presently disclosed systems and methods are susceptible to embodiment in many different forms, there are shown in the drawings and will herein be described in detail several specific embodiments with the understanding that the present disclosure is to be considered as an exemplification of the principles of the presently disclosed systems and methods and is not intended to limit the disclosure systems and methods to the embodiments illustrated.

While specific embodiments of, and examples for, the system are described above for illustrative purposes, various equivalent modifications are possible within the scope of the system, as those skilled in the relevant art will recognize. For example, while processes or steps are presented in a given order, alternative embodiments may perform routines having steps in a different order, and some processes or steps may be deleted, moved, added, subdivided, combined, and/or modified to provide alternative or sub-combinations. Each of these processes or steps may be implemented in a variety of different ways. Also, while processes or steps are at times shown as being performed in series, these processes or steps may instead be performed in parallel or may be performed at different times.

While various embodiments have been described above, it should be understood that they have been presented by way of example only and not limitation. The descriptions are not intended to limit the scope of the present technology to the particular forms set forth herein. To the contrary, the present descriptions are intended to cover such alternatives, modifications, and equivalents as may be included within the spirit and scope of the present technology as appreciated by one of ordinary skill in the art. Thus, the breadth and scope of a preferred embodiment should not be limited by any of the above-described exemplary embodiments.

What is claimed is:

1. A method of assessing a degradation activity of a test compound, comprising:
contacting a cell comprising a fusion protein with the test compound, wherein the fusion protein comprises a target protein of interest and a DNA-binding protein domain;
incubating the cell under conditions promoting degradation of the target protein of interest;
lysing the cell to form cell lysate;
linking the fusion protein present in the lysate to a nucleic acid oligomer tag;
incubating the cell lysate in the presence of a ligand immobilized on a solid support, wherein the ligand is previously known to bind to the target protein of interest;
detecting an expression level of the target protein of interest bound to the ligand by detecting the nucleic acid oligomer tag in the presence of the test compound; and
comparing to an expression level of the target protein of interest in the absence of the test compound, wherein a decrease in the expression level of the target protein of interest in the presence of the test compound as compared to the absence of the test compound indicates the test compound promotes degradation of the target protein of interest.

2. The method of claim 1, wherein the test compound is not previously known to degrade the target protein of interest.

3. The method of claim 1, wherein the target protein of interest and the nucleic acid oligomer tag are not the same.

4. The method of claim 1, further comprises:
contacting the cell comprising the fusion protein with a control compound, the control compound is previously known to degrade the target protein of interest;
lysing the cell to form a cell lysate;
incubating the cell lysate in the presence of a ligand immobilized on the solid support;
detecting the expression level of the target protein of interest by detecting the nucleic acid oligomer tag; and
comparing the expression level of the target protein of interest bound to the ligand in the absence of the control compound to the expression level of the target protein of interest bound to the ligand in the presence of the control compound, wherein a decrease in the expression level of the target protein of interest in the presence of the control compound, as compared to the expression level of the target protein of interest in the absence of the control compound, indicates that the control compound promotes degradation of the target protein of interest.

5. The method of claim 4, wherein the ligand immobilized on the solid support is a small molecule, a peptide, or a protein.

6. The method of claim 4, wherein the test compound tags the target protein of interest for degradation by inducing binding between the target protein of interest and a ligase, wherein the ligase is an E3 ligase.

7. The method of claim 6, wherein the binding of the ligase tags the target protein of interest for degradation within a proteosome.

8. The method of claim 1, wherein the test compound is a protein, a heterobifunctional degrader, a molecular glue, or a small molecule.

9. The method of claim 4, wherein the amount of the target protein of interest is measured by quantitative PCR.

10. The method of claim 4, wherein the method further comprises contacting the cell with an inhibitor is previously known to inhibit protein degradation.

11. A method of assessing a degradation activity of a test compound, comprising:
contacting a cell comprising a fusion protein simultaneously with the test compound and an inhibitor is previously known to inhibit protein degradation, wherein the fusion protein comprises a target protein of interest and a DNA-binding protein domain;
incubating the cell under conditions promoting degradation of the target protein of interest;
lysing the cell to form a cell lysate;
linking the fusion protein present in the lysate to a nucleic acid oligomer tag;
incubating the cell lysate in the presence of a ligand immobilized on a solid support, wherein the ligand is known to bind to the target protein of interest;
detecting an expression level of the target protein of interest bound to the ligand by detecting the nucleic acid oligomer tag in the presence of the test compound and the inhibitor; and
comparing with an expression level of the target protein of interest in the absence of the inhibitor and presence of test compound, wherein an increase in the expression level of the target protein of interest in the presence of the inhibitor and the test compound as compared to in the absence of the inhibitor and presence of test compound, indicates that the test compound reduces the expression level of the target protein of interest by promoting protein degradation.

12. The method of claim 11, wherein the test compound is not previously known to degrade the target protein of interest.

13. The method of claim 11, wherein the ligand immobilized on the solid support is a small molecule, a peptide, or a protein.

14. The method of claim 11, wherein the test compound is a protein, a heterobifunctional degrader, a molecular glue, or a small molecule.

15. The method of claim 11, wherein the inhibitor is previously known to inhibit the degradation of the target protein of interest.

16. The method of claim 15, wherein the inhibitor is a ubiquitination inhibitor, a protein inhibitor, or a proteosome inhibitor.

17. The method of claim 11, wherein the target protein of interest and the nucleic acid tag are not the same.

18. The method of claim 11, wherein the test compound tags the target protein of interest for degradation by inducing binding between the target protein of interest and a ligase, wherein the ligase is an E3 ligase.

19. The method of claim 11, wherein the binding of the ligase tags the target protein of interest for degradation within a proteosome.

20. The method of claim 11, wherein the method further comprises contacting an expression vector comprising the fusion protein with the test compound.
